(19) United States
(12) Patent Application Publication
(10) Pub. No.: US 2025/0331888 A1
(43) Pub. Date: Oct. 30, 2025

(54) ECHOGENIC PATTERN FOR COMPONENT DISTINGUISHABILITY

(71) Applicant: BOSTON SCIENTIFIC SCIMED, INC., Maple Grove, MN (US)

(72) Inventors: Laine Monroe PALMER, West Barnstable, MA (US); Daniel Dina LON, Lynn, MA (US); Zachary William FRIEDLANDER, Arlington, MA (US); Jordan Thomas HANAHAN, Natick, MA (US)

(21) Appl. No.: 19/079,032

(22) Filed: Mar. 13, 2025

Related U.S. Application Data
(60) Provisional application No. 63/640,615, filed on Apr. 30, 2024.

(51) Int. Cl. A61B 17/34; A61B 17/00; A61B 34/20

(52) U.S. Cl. CPC A61B 17/3421; A61B 17/00234; A61B 34/20; A61B 2017/00818; A61B 2017/3413; A61B 2034/2065

(57) ABSTRACT

A system for accessing a biliary duct includes an access cannula and a needle. The cannula extends longitudinally from a proximal end to a distal end and includes a channel extending therethrough. A distal portion of the cannula includes an echogenic pattern defined via echogenic segments, each of which are separated from one another via a space so that the echogenic pattern is configured to produce alternating bright and dark regions, when visualized under ultrasound guidance. The needle extends longitudinally from a proximal end to a distal end, and sized, shaped, and configured to be received within the channel of the cannula. The distal end of the needle includes a sharp tip producing a localized bright spot, under an ultrasound guidance, which is distinguishable from the bright and dark regions created via the echogenic pattern of the cannula.

### PRIORITY CLAIM

The present disclosure claims priority to U.S. Provisional Patent Application Ser. No. 63/640,615 filed Apr. 30, 2024; the disclosure of which is incorporated herewith by reference

### FIELD

The present disclosure relates to Endoscopic Ultrasound (EUS) devices including an echogenic pattern to enhance visibility.

### BACKGROUND

An Endoscopic Ultrasound (EUS) access procedure is a minimally invasive procedure performed with a specialized endoscope that uses high frequency soundwaves to visualize, for example, the digestive (gastrointestinal) tract and other nearby structures. According to one application, EUS is used to facilitate direct biliary drainage when, for example, a traditional endoscopic retrograde cholangiopancreatology (ERCP), which utilizes contrast dye and X-rays to identify and treat blockages within the ducts via the papilla, has failed. EUS may be used to visualize the ducts directly through the gastric wall, facilitating puncture with a needle to gain guidewire access to the common bile, hepatic or pancreatic ducts. Thus, the success of EUS access procedures may be impacted by the visibility of the device.

In particular, successful EUS access procedures depend on an initial duct puncture and access cannula stability within the duct while passing a guidewire to the target site. However, the use of needles to puncture ducts, particularly smaller ducts, poses an inherent risk for a through and through puncture in which the needle tip not only pierces the near wall of the duct, but passes through a far wall of the duct as well. Thus, the position of the sharp tip of the needle is critical to ensure a safe puncture that passes through the near wall of the duct to gain access thereto, but which is not through and through. The position of the access cannula is critical to maintain access to the duct to permit the performance of downstream procedural steps (e.g., guidewire placement or fistula creation). While various techniques have been developed to improve echogenic visibility (i.e., the ability to reflect an echo of sound waves to enhance visibility of the device under ultrasound guidance) for devices configured primarily for biopsy or aspiration, it remains challenging to ensure accurate placement of an EUS access device, as EUS access devices include multiple components that are difficult to distinguish from one another under EUS guidance.

### SUMMARY

The present disclosure relates to a system for accessing a biliary duct. The system includes an access cannula extending longitudinally from a proximal end to a distal end and including a channel extending therethrough, a distal portion of the access cannula including an echogenic pattern defined via echogenic segments, each of which are separated from one another via a space so that the echogenic pattern is configured to produce alternating bright and dark regions, when visualized under ultrasound guidance. In addition, the system includes a needle extending longitudinally from a proximal end to a distal end, and sized, shaped and configured to be received within the channel of the access cannula, the distal end of the needle including a sharp tip producing a localized bright spot, under an ultrasound guidance, that is distinguishable from the bright and dark regions created via the echogenic pattern of the access cannula.

In an embodiment, each of the echogenic segments include a plurality of markings extending along a portion of a length of an exterior surface of the access cannula.

In an embodiment, each of the markings is configured as a circumferential ring extending about the access cannula.

In an embodiment, each of the markings is configured as a depression extending into the exterior surface along a curve.

In an embodiment, each of the markings is configured as a roughened portion along the exterior surface.

In an embodiment, a distal-most one of the echogenic segments is offset from the distal end of the access cannula via a length of at least 3.0 mm.

In an embodiment, the echogenic segments extend along a length of between 2.5 mm and 12.5 mm of the access cannula.

In an embodiment, the space extending between each of the echogenic segments extends along a length of between 2.0 mm and 12.0 mm of the access cannula.

In an embodiment, the distal end of the access cannula is tipped to include a tapered surface which produces a bright region, when visualized under the ultrasound guidance.

In an embodiment, the echogenic pattern is formed via a first echogenic material that is one of swaged and reflowed along an exterior surface of a second non-echogenic material forming a base of the access cannula, along predetermined segments that are separated from one another.

In an embodiment, the first echogenic material is a glass-filled polymer and the second non-echogenic material is a polymer extrusion.

In an embodiment, the access cannula is formed of an echogenic material, the spaces between echogenic segments formed via one of a non-echogenic heat shrink and jacket wrapped around portions of the access cannula.

In addition, the present disclosure relates to an endoscopic device. The device includes an access cannula sized, shaped and configured to be inserted through a working channel of an endoscope, the access cannula extending along a longitudinal axis from a proximal end to a distal end and including an echogenic pattern along a distal portion thereof, the echogenic pattern defined via echogenic segments, each of which are separated from one another via a space so that the echogenic pattern is configured to produce alternating bright and dark regions, when visualized under ultrasound guidance. In addition, the device includes a needle sized, shaped and configured to be slidably received within a channel of the access cannula, the needle extending longitudinally from a proximal end to a sharp distal tip which produces a localized bright spot, when visualized under an ultrasound guidance, so that the sharp distal tip is distinguishable from the bright and dark regions created via the echogenic pattern of the access cannula.

In an embodiment, each of the echogenic segments include a plurality of circumferential rings etched into an exterior surface of the access cannula.

In an embodiment, the access cannula is formed of a first echogenic material defining the echogenic segments and a second non-echogenic material defining the spaces therebetween.

In addition, the preset disclosure relates to a method for accessing a biliary duct. The method includes comprising: inserting an endoscope to target area within a stomach; inserting an access cannula, with a needle received therein, through a working channel of the endoscope to a target area within a body, under an endoscopic ultrasound image guidance (EUS); pressing a distal end of the access cannula against a portion of a wall of the stomach adjacent a target wall of a target duct to be accessed so that a sharp tip of the needle pierces the stomach wall, the sharp tip of the needle visible as a localized bright spot under the EUS; moving the access cannula and the needle toward the target wall of the target duct and puncturing the target wall of the target duct via the sharp tip of the needle, the access cannula visually distinguishable from the sharp tip via an echogenic pattern extending along a distal portion of the access cannula; and moving the access cannula distally over the sharp tip so that the distal end of the access cannula passes through the puncture to be received within the target duct.

In an embodiment, the echogenic pattern is defined via echogenic segments, each of which are separated from one another via a space so that the echogenic pattern is configured to produce alternating bright and dark regions, when the access cannula is visualized under EUS.

In an embodiment, each of the echogenic segments is formed via a plurality of circumferential rings etched into an exterior surface of the access cannula.

In an embodiment, the echogenic segments are formed via a first echogenic material that is one of swaged and reflowed along an exterior surface of a second non-echogenic material forming a base of the access cannula.

In an embodiment, the access cannula is formed of an echogenic material, the spaces between echogenic segments formed via one of a non-echogenic heat shrink and jacket wrapped around portions of the access cannula.

### BRIEF DESCRIPTION

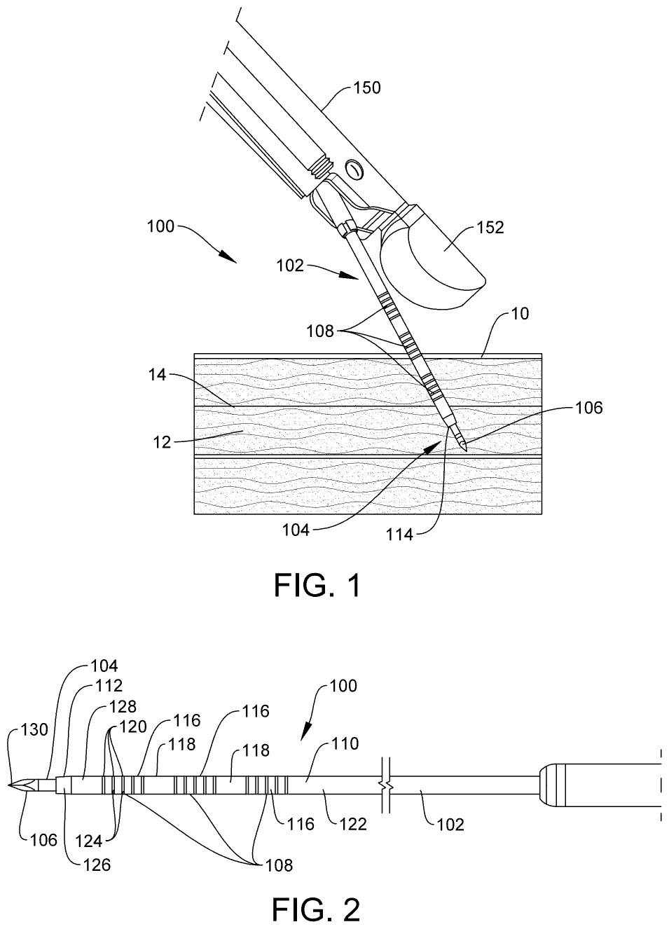

FIG. 1 shows a side view of a system according to an exemplary embodiment of the present disclosure;

FIG. 2 shows a side view of a distal portion of the system according to FIG. 1;

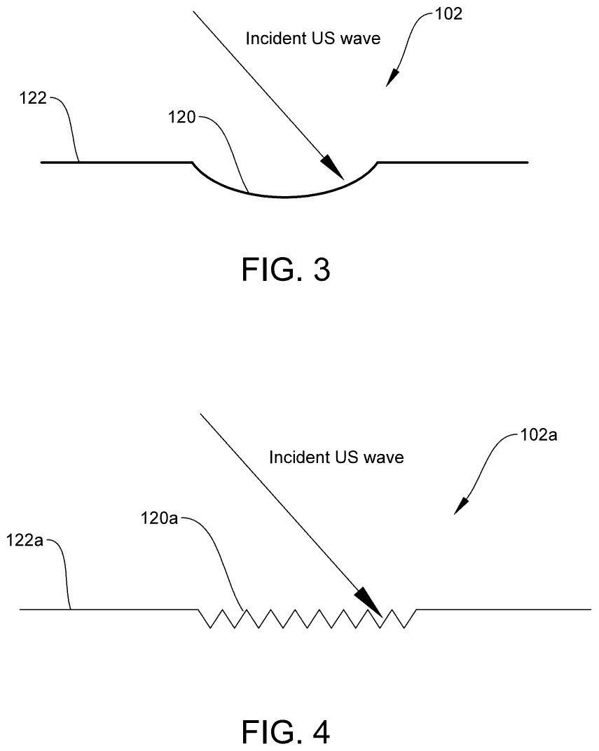

FIG. 3 shows an enlarged partial side view of a marking along an access cannula according to the system of FIG. 1;

FIG. 4 shows an enlarged partial side view of a marking along an access cannula according to an alternate embodiment; and

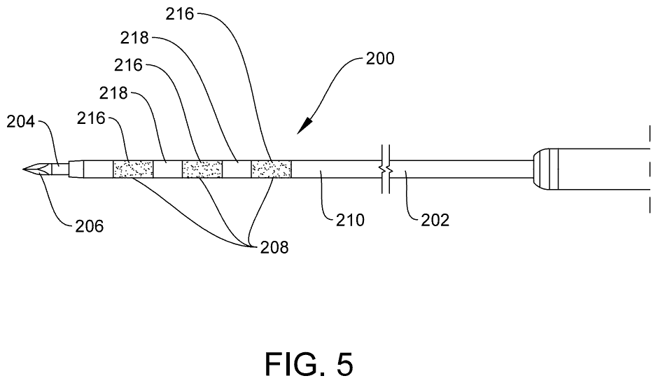

FIG. 5 shows a side view of a system according to another exemplary embodiment of the present disclosure.

### DETAILED DESCRIPTION

The present disclosure may be further understood with reference to the following description and appended drawings, wherein like elements are referred to with the same reference numerals. The present disclosure relates to an endoscopic needle system and, in particular, relates to an EUS needle system including an echogenic pattern for component distinguishability. Exemplary embodiments of the present disclosure describe a system comprising an access cannula including an echogenic pattern along a distal portion thereof. The echogenic pattern is configured to distinguish, under ultrasonic guidance, the access cannula from a needle received therein to ensure accurate placement/positioning of the components of the system. It should be noted that although the exemplary embodiments specifically describe a needle system for accessing a biliary duct for, for example, a biliary drainage procedure, it will be understood by those of skill in the art that the exemplary system may be utilized for any of a variety of procedures in which access to a duct or organ is desired. It should also be noted that the terms “proximal” and “distal,” as used herein, are intended to refer to a direction toward (proximal) and away from (distal) a user of the device (e.g., physician).

As shown in FIGS. 1-4, a needle access system 100 for treating, for example, a biliary duct, according to an exemplary embodiment of the present disclosure comprises an access cannula 102 and a needle 104 housed therein. The needle 104 is slidably received within the access cannula 102 and includes a sharp tip 106 configured to puncture a wall of, for example, a target duct. The access cannula 102 includes an echogenic pattern 108 along a distal portion 110 thereof. In an exemplary embodiment, the echogenic pattern 108 is configured to produce alternating bright and dark regions, when under EUS guidance, to enhance visibility of the access cannula 102 while also visually distinguishing the access cannula 102 from the needle 104 and, in particular, the sharp tip 106. In particular, the alternating bright and dark regions clearly indicate a position of the access cannula 102 relative to the sharp tip 106. Thus, a user (e.g., physician) is able to confirm an accurate positioning of both the sharp tip 106 and the access cannula 102 relative to one another and to the target duct.

Upon puncturing of the duct wall via the sharp tip 106 of the needle 104, the access cannula 102 may be inserted through the puncture in the duct wall so that a distal end 112 of the access cannula 102 passes through the puncture into the target duct. In an exemplary embodiment, the needle 104 may be received within the access cannula 102 so that the sharp tip 106 extends distally therefrom via a selected distance. In this embodiment, the needle 104 and the access cannula 102 are moved together so that, as the needle 104 is moved distally relative to, for example, an endoscope, to puncture the target duct, the access cannula 102 is moved distally along with it, just slightly proximally thereof.

It will be understood by those of skill in the art that the distance via which the sharp tip 106 extends distally from the access cannula 102 may be selected to reduce the likelihood of a through and through puncture as a distal end 112 of the access cannula 102 is received within the target duct. In another exemplary embodiment, the access cannula 102 may be longitudinally movable relative to the needle 104 so that, upon puncturing of the target duct via the sharp tip 106, the access cannula 102 may be moved distally thereover so that the distal end 112 of the access cannula 102 is received within the target duct. Once the access cannula 102 has entered the target duct, the needle 104 may be removed therefrom so that other devices and/or tools such as, for example, a guidewire, may be inserted into the target duct via the channel of the access cannula 102. In an exemplary embodiment, as shown in FIG. 1, the system 100 may be inserted to a target area within a patient body via, for example, a working channel of a flexible endoscope 150 that has been previously placed in the desired position after passing along a tortuous path (e.g., along a portion of the alimentary canal).

As shown in FIG. 2, the access cannula 102 is a flexible hollow member that extends longitudinally from a proximal end (not shown) to the distal end 112 and includes a channel 114 extending longitudinally therethrough, from the proximal end to the distal end 112. In an exemplary embodiment, the access cannula 102 includes a tipped—e.g., tapered-distal end 112 configured to facilitate insertion of the distal end 112 through the puncture and into the target duct. It will be understood by those of skill in the art that a tapered surface 126 of the tipped distal end 112 creates a naturally echogenic portion of the access cannula 102 that is visible to the user under EUS. As described above, the distal portion 110 of the access cannula 102 also includes the echogenic pattern 108 therealong, where the echogenic pattern 108 is specifically configured to produce areas of alternating bright and dark regions when visualized under EUS guidance. This permits a user to observe a position and/or orientation of the access cannula 102 relative to the sharp tip 106.

According to an exemplary embodiment, the echogenic pattern 108 includes a plurality of echogenic segments 116, separated from adjacent echogenic segments 116 longitudinally along the access cannula 102 via spaces 118 selected therebetween. As would be understood by those skilled in the art, under EUS guidance, each of the spaces 118 produces a dark region between adjacent echogenic segments 116. In an exemplary embodiment, a first, distal-most one of the echogenic segments 116 is offset from the distal end 112 by a space 128 to create a dark region, visible under EUS, between the tipped distal end 112 and the start of the echogenic pattern 108. In an exemplary embodiment, the distal-most echogenic segment 116 is separated from the distal end 112 of the access cannula 102 by a distance of approximately 3.0 mm. It will be understood by those of skill in the art, however, that a length of this offset (the length of the space 128) may be varied so long as the dark region separating a bright region produced at the distal end 112 of the access cannula 102 from the distal-most echogenic segment 116 is visible under EUS.

Similarly, the size of each of the spaces 118 extending between adjacent echogenic segments 116 is selected to produce a visible dark region between adjacent ones of the echogenic segments 116. It will be understood by those of skill in the art that these dark regions produced by the spaces 118, when under EUS, may enhance the brightness of the echogenic segments 116, facilitating ready visualization of a position and/or orientation of the distal portion 110 of the access cannula 102. In an exemplary embodiment, each of the spaces 118 between the echogenic segments 116 has a length of approximately 2.0 mm. It will be understood by those of skill in the art, however, that the spaces 118 may have any of a variety of lengths so long as the spaces 118 are configured to produce dark regions between the echogenic segments 116, under EUS, as described above. In an exemplary embodiment, each of the spaces 118 may have a length ranging from between 1.5 mm to 12.0 mm.

It will be understood by those of skill in the art, however, that the lengths of the spaces 118 need not be equal to one another and the lengths of each of the spaces 118 may vary relative to one another. As indicated above, the length of the various spaces 118 may vary so long as each of the spaces 118 has a length sufficient to produce an easily recognizable dark region between the bright regions of the echogenic segments 116. In an exemplary embodiment, the echogenic segments 116 are equally spaced from one another. In another embodiment, the length of the spaces 118 differ from one another along a length of distal portion 110 of the access cannula 102.

In an exemplary embodiment, each of the echogenic segments 116 includes a plurality of marking 120, each of which is, for example, etched or ground into an exterior surface 122 of the access cannula 102. In one example, the markings 120 form a series of circumferential rings 124, each of which extends about at least a portion of the circumference of the access cannula 102. The circumferential rings 124 together produce a bright region, when visualized under EUS. In an exemplary embodiment, each of the echogenic segments 116 includes the same number of circumferential rings 124 although the number of circumferential rings 124 in each of the echogenic segments 116 can vary to, for example, identify different locations on the distal portion 110.

In one example, each of the echogenic segments 116 includes five circumferential rings 124 with a totality of the circumferential rings 124 extending along a length of the access cannula 102 measuring approximately 2.5 mm, the distal portion 110 along which an overall pattern extends (e.g., from the distal end 112 to a proximal end of the proximal-most echogenic segment 116) having a length of approximately 1.5 cm. In another exemplary embodiment, each of the echogenic segments 116 may have a length of up to 12.5 mm so that the length of the distal portion 110 along which the overall pattern extends may have a length of up to 6.5 cm. It will be understood by those of skill in the art, however, that the echogenic segments 116 may extend along a variety of different lengths of the access cannula 102 and may be comprised of any of a number of the markings 120 or the circumferential rings 124 so long as the echogenic segments 116 produce a bright region that is readily visible to the user, and distinct from the dark regions produced via the spaces 118.

In one exemplary embodiment, a length of each of the echogenic segments 116 may substantially correspond to a length of the spaces 118 therebetween so that the bright and dark regions produced thereby, under EUS, may be roughly the same length along the access cannula 102. Thus, the visual cue under ultrasound may be a recognizable and/or reliable pattern. In another exemplary embodiment, however, the lengths of the echogenic segments 116 may be different from the lengths of the spaces 118 so that the bright regions, under EUS, extend along a longer length of the access cannula 102 than the dark regions. In yet another exemplary embodiment, however, the lengths of the echogenic segments 116 may vary relative to one another and relative to the lengths of the spaces 118.

Although the markings 120 are shown and described as circumferential rings 124 equally spaced from one another, it will be understood by those of skill in the art that the access cannula 102 may include any of a number of markings 120, each of which may have any of a variety of configurations. In one example, each marking 120 extends about only a part of the circumference of the access cannula 102 (i.e., the markings 120 are not fully circumferential). In another example, the markings 120 are separated from one another by varying distances. In yet another example, the different markings 120 are configured to extend along varying lengths of the access cannula 102.

As described above, each of the markings 120 may be etched into the exterior surface 122 of the access cannula 102. The etched markings 120 create a depression, as shown in FIG. 3, formed along, for example, a substantially perpendicular exterior surface 122 of the access cannula 102 Each of these depressions is configured to reflect sound waves back to an EUS transducer 152 of, for example, the endoscope 150, at a variety of incident angles such that the access cannula 102 is echogenic while in a variety of positions relative to the EUS transducer 152. In one exemplary embodiment, the depression may be configured as a groove extending into the exterior surface 122, the groove extending along a curved surface.

It will be understood by those of skill in the art, however, that each of the markings 120 may take any of a variety of configurations so long as each marking 120 is configured to reflect sound waves, as described above sufficiently to make the marked areas visible to a user under EUS. For example, the depressions of any or all of the markings 120 may include any of a variety of angled and/or curved surfaces. In another example, as shown in FIG. 4, each marking 120a may be etched into an exterior surface 122a of an access cannula 102a to form a roughened portion of the surface which is configured to reflect sound waves back to the EUS transducer 152, as described above.

In an exemplary embodiment, the echogenic pattern 108 along the access cannula 102 may be comprised of three echogenic segments 116 therealong separated by two spaces 118. It will be understood by those of skill in the art that the echogenic pattern 108 may include any of a number of echogenic segments 116 so long as an orientation/position of the access cannula 102 relative to the needle 104 and/or the target duct is easily discernible therefrom, when under EUS guidance. The access cannula 102 may be formed of any of a variety of materials including, for example, polyetheretherketone (PEEK). The markings 120 may be achieved via, for example, a laser etching or other mechanical processing thereof.

It will be understood by those of skill in the art, however, that the access cannula 102 may be formed of any of a variety of medical grade materials so long as the access cannula 102 is configured to provide access to a target duct, as described above, and to be etched to include markings 120, as described above. In another exemplary embodiment, the above-described echogenic pattern 108 may be similarly imparted on a non-naturally echogenic material through other techniques such as, for example, grit blasting or other etching forms, to create a similar distinguishing effect under EUS.

The needle 104 extends longitudinally from a proximal end (not shown) to a distal end 130 including the sharp tip 106. The needle 104 is sized, shaped and configured to be slidably received within the channel 114 of the access cannula 102. In an exemplary embodiment, a length of the needle 104 and/or the sharp tip 106 is selected so that the sharp tip 106 can be extended distally beyond the distal end 112 of the access cannula 102 by a desired distance to puncture a near wall of the target duct, without also puncturing a far wall thereof. In one example, the sharp tip 106 is extendible distally from the distal end 112 of the access cannula 102 by a distance of 2.5 mm. Upon puncturing of the wall of the target duct and insertion of the distal end 112 of the access cannula 102 into the target duct, however, the needle 104 may be removed from the channel 114 of the access cannula 102.

The sharp tip 106 may have any of a variety of configurations, so long as the sharp tip 106 is configured to pierce the target wall. In one embodiment, the sharp tip 106 may be configured as a trocar tip. It will be understood by those of skill in the art that the configuration of the sharp tip 106 causes the sharp tip 106 to be naturally echogenic so that is visible as a localized bright spot, under EUS guidance. While the sharp tip 106 may be visualized as a localized bright spot, the access cannula 102, as described above, produces alternating bright and dark regions as described above. Thus, the user may easily distinguish the access cannula 102 from the sharp tip 106. As would be understood by those skilled in the art, an ultrasound image of a needle including the echogenic pattern 108 and the distal end 112 of the access cannula 102 will show bright areas at the locations 108 that are separated from and clearly distinguishable from the bright region produced via the sharp tip 106.

According to an exemplary method, the system 100 may be utilized to access the target duct to provide treatment thereto. An insertion device such as, for example, the endoscope 150 is inserted through a body lumen (e.g., into the mouth, through the esophagus into the stomach) until a distal end thereof is positioned in a target area of a patient body (e.g., within the stomach of a patient, proximate a target duct to be accessed), as shown in FIG. 1. When the endoscope 150 has been positioned as desired, the system 100—with the needle 104 received within the access cannula 102—is inserted through a working channel of the endoscope 150 until the distal end 112 of the access cannula 102 extends out of the working channel to contact a desired site on the stomach wall 10 (e.g., along a portion of the stomach wall 10 corresponding to an area of the target duct 12 to be accessed). In an exemplary embodiment, the sharp tip 106 of the needle 104 extends distally past the distal end 112 to pierce and/or puncture the wall of the stomach wall 10. The needle 104 and the access cannula 102 may, together, be moved through the stomach wall 10 toward the wall of the target duct 12.

The user may continue to move the access cannula 102 and the needle 104 distally until the sharp tip 106 of the needle 104 punctures a near wall 14 of the target duct 12. As described above, a length of the distal end 130 of the needle 104 extending distally past the distal end 112 of the access cannula 102 may be selected to prevent a through and through puncture of the target duct 12. Upon piercing of the near wall of the target duct 12, the access cannula 102 is moved through the puncture in the near wall 14 so that the distal end 112 is inserted into the target duct 12 via the puncture formed through the wall 14 thereof.

Once the target duct 12 has been accessed via the access cannula 102, the needle 104 is removed from the access cannula 102, leaving the access cannula 102 in place for subsequent treatment. In an exemplary embodiment, for example, a guidewire is passed through the access cannula 102 into the target duct 12 so that a catheter or other device may be inserted thereover into the target duct to facilitate drainage of the target duct. The method described above is an exemplary method for facilitating a draining of target duct. It will be understood by those of skill in the art, however, that the system 100 may be utilized for other applications in which access of a duct or other hollow organ may be desired.

Although the access cannula 102 of the system 100 is described as including an echogenic pattern 108 formed via the circumferential rings 124 etched into an exterior surface 122 thereof, it will be understood by those of skill in the art that an echogenic pattern along an access cannula may be created via alternate mechanical treatments thereof, so long as the echogenic pattern is configured to produce alternating bright and dark regions, under EUS, as described above.

According to another exemplary embodiment, as shown in FIG. 5, a system 200 may be substantially similar to the system 100 described above, comprising an access cannula 202 having an echogenic pattern 208 along a distal portion 210 thereof. The echogenic pattern 208 forms a substantially similar band pass design, which produces alternating bright and dark regions, when viewed under EUS. Similarly to the system 100, the system 200 incudes a needle 204 received within the access cannula 202, the needle 204 including a sharp tip 206 for piercing a wall of a target duct to facilitate insertion of a distal end 212 of the access cannula 202 therein.

The echogenic pattern 208 along the access cannula 202 in this embodiment, however, may be imparted by the provision of two materials having differing echogenic properties so that alternating bands of these materials generate the same contrast in brightness in the image as generated above by the echogenic segments 116 and the spaces 118. That is, a first material will generate bright locations in the image corresponding to the echogenic segments 216 while bands of the other material create dark regions (non-echogenic spaces 218) extending therebetween.

In one exemplary embodiment, the two materials include a more echogenic material forming echogenic segments 216 and a non-naturally echogenic base material forming a remainder of the access cannula 202 including the spaces 218 between the echogenic segments 216. For example, glass-filled polymer is more echogenic than a virgin polymer extrusion because it has a rougher surface texture which facilitates redirection of scattered waves back to, for example, an EUS transducer. Thus, in an exemplary embodiment, glass-filled polymer may be swaged or reflowed onto the virgin polymer extrusion's outer surface in intentionally sized echogenic segments 216 so that they are separated from one another via intentionally sized spaces 218 formed of the virgin polymer extrusion with no glass-filled polymer coating.

The varying materials of the echogenic segments 216 and spaces 218 produce a pattern or bright and dark regions similar to that as described above with respect to system 100, when visualized under EUS. For example, lengths/sizes of the echogenic segments 216 and spaces 218 may be substantially similar to the echogenic segments 116 and spaces 118, respectively, as described above with respect to the system 100. According to another exemplary embodiment, a metal or other material having a higher acoustic impedance compared to the base material of the access cannula 202 may be swaged along the exterior surface of the access cannula 202, as described above, to form the echogenic pattern 208.

The above-described embodiment assumes that the sharp tip 206 of the needle 204 is highly echogenic while the access cannula 202 is not naturally echogenic so that the echogenic pattern 208 created thereby generates a visually distinguishable feature. According to another exemplary embodiment, in which the access cannula 202 is also highly echogenic, the band pass design of the echogenic pattern 208 described above may be imparted by creating areas of less to no echogenicity—e.g., the dark regions-via, for example, a heat shrink or jacket wrapped around portions of the access cannula 202. In other words, the spaces 218 between the echogenic segments 216, in this embodiment, are formed by wrapping or covering portions of the echogenic material of the access cannula 202 with a non-echogenic material.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather, modifications are also covered within the scope of the present invention as defined by the appended claims.

1-15. (canceled)

16. A system for accessing a biliary duct, comprising:
an access cannula extending longitudinally from a proximal end to a distal end and including a channel extending therethrough, a distal portion of the access cannula including an echogenic pattern defined via echogenic segments, each of which are separated from one another via a space so that the echogenic pattern is configured to produce alternating bright and dark regions, when visualized under ultrasound guidance; and
a needle extending longitudinally from a proximal end to a distal end, and sized, shaped and configured to be received within the channel of the access cannula, the distal end of the needle including a sharp tip producing a localized bright spot, under an ultrasound guidance, that is distinguishable from the bright and dark regions created via the echogenic pattern of the access cannula.

17. The system of claim 16, wherein each of the echogenic segments include a plurality of markings extending along a portion of a length of an exterior surface of the access cannula.

18. The system of claim 17, wherein each of the markings is configured as a circumferential ring extending about the access cannula.

19. The system of claim 17, wherein each of the markings is configured as a depression extending into the exterior surface along a curve.

20. The system of claim 17, wherein each of the markings is configured as a roughened portion along the exterior surface.

21. The system of claim 17, wherein a distal-most one of the echogenic segments is offset from the distal end of the access cannula via a length of at least 3.0 mm.

22. The system of claim 16, wherein the echogenic segments extend along a length of between 2.5 mm and 12.5 mm of the access cannula.

23. The system of claim 16, wherein the space extending between each of the echogenic segments extends along a length of between 2.0 mm and 12.0 mm of the access cannula.

24. The system of claim 16, wherein the distal end of the access cannula is tipped to include a tapered surface which produces a bright region, when visualized under the ultrasound guidance.

25. The system of claim 16, wherein the echogenic pattern is formed via a first echogenic material that is one of swaged and reflowed along an exterior surface of a second non-echogenic material forming a base of the access cannula, along predetermined segments that are separated from one another.

26. The system of claim 25, wherein the first echogenic material is a glass-filled polymer and the second non-echogenic material is a polymer extrusion.

27. The system of claim 16, wherein the access cannula is formed of an echogenic material, the spaces between echogenic segments formed via one of a non-echogenic heat shrink and jacket wrapped around portions of the access cannula.

28. An endoscopic device, comprising:
an access cannula sized, shaped and configured to be inserted through a working channel of an endoscope, the access cannula extending along a longitudinal axis from a proximal end to a distal end and including an echogenic pattern along a distal portion thereof, the echogenic pattern defined via echogenic segments, each of which are separated from one another via a space so that the echogenic pattern is configured to produce alternating bright and dark regions, when visualized under ultrasound guidance; and
a needle sized, shaped and configured to be slidably received within a channel of the access cannula, the needle extending longitudinally from a proximal end to a sharp distal tip which produces a localized bright spot, when visualized under an ultrasound guidance, so that the sharp distal tip is distinguishable from the bright and dark regions created via the echogenic pattern of the access cannula.

29. The device of claim 28, wherein each of the echogenic segments include a plurality of circumferential rings etched into an exterior surface of the access cannula.

30. The device of claim 28, wherein the access cannula is formed of a first echogenic material defining the echogenic segments and a second non-echogenic material defining the spaces therebetween.

31. A method for accessing a biliary duct, comprising:
inserting an endoscope to target area within a stomach;
inserting an access cannula, with a needle received therein, through a working channel of the endoscope to a target area within a body, under an endoscopic ultrasound image guidance (EUS);
pressing a distal end of the access cannula against a portion of a wall of the stomach adjacent a target wall of a target duct to be accessed so that a sharp tip of the needle pierces the stomach wall, the sharp tip of the needle visible as a localized bright spot under the EUS;
moving the access cannula and the needle toward the target wall of the target duct and puncturing the target wall of the target duct via the sharp tip of the needle, the access cannula visually distinguishable from the sharp tip via an echogenic pattern extending along a distal portion of the access cannula; and
moving the access cannula distally over the sharp tip so that the distal end of the access cannula passes through the puncture to be received within the target duct.

32. The method of claim 31, wherein the echogenic pattern is defined via echogenic segments, each of which are separated from one another via a space so that the echogenic pattern is configured to produce alternating bright and dark regions, when the access cannula is visualized under EUS.

33. The method of claim 32, wherein each of the echogenic segments is formed via a plurality of circumferential rings etched into an exterior surface of the access cannula.

34. The method of claim 32, wherein the echogenic segments are formed via a first echogenic material that is one of swaged and reflowed along an exterior surface of a second non-echogenic material forming a base of the access cannula.

35. The method of claim 32, wherein the access cannula is formed of an echogenic material, the spaces between echogenic segments formed via one of a non-echogenic heat shrink and jacket wrapped around portions of the access cannula.
